(19) United States
(12) Patent Application Publication
(10) Pub. No.: US 2025/0391564 A1
(43) Pub. Date: Dec. 25, 2025

(54) APPARATUS AND METHOD FOR SUPPORTING DIAGNOSIS OF INTRAVENTRICULAR HEMORRHAGE AND EARLY DEATH WITHIN A WEEK IN VERY LOW BIRTH WEIGHT INFANTS BASED ON DEEP LEARNING

(71) Applicants: JEONBUK NATIONAL UNIVERSITY HOSPITAL, Jeonbuk-do (KR); INDUSTRIAL COOPERATION FOUNDATION JEONBUK NATIONAL UNIVERSITY, Jeonbuk-do (KR)

(72) Inventors: HYUN HO KIM, Jeonbuk-do (KR); MYOUNG-HWAN KO, Jeonbuk-do (KR)

(21) Appl. No.: 18/883,128

(22) Filed: Sep. 12, 2024

(30) Foreign Application Priority Data
Jun. 20, 2024 (KR) 10-2024-0080192

(51) Int. Cl. G16H 50/20; A61B 5/02; G16H 10/60

(52) U.S. Cl. CPC G16H 50/20; G16H 10/60

(57) ABSTRACT

A method for supporting diagnosis of intraventricular hemorrhage and early death within a week in very low birth weight infants based on deep learning includes a data collection step, a data preprocessing step, a learning step of training a prenatal prediction model, training a birth prediction model and training a postnatal prediction model, and a diagnosis step of, when a diagnosis target and diagnosis time are determined, selecting one prediction model based on the diagnosis time, analyzing medical information of the diagnosis target through the selected prediction model, and predicting and outputting the intraventricular hemorrhage diagnosis result.

### CROSS-REFERENCE TO RELATED APPLICATION AND CLAIM OF PRIORITY

This application claims the benefit under 35 USC § 119 of Korean Patent Application No. 10-2024-0080192, filed on Jun. 20, 2024, in the Korean Intellectual Property Office, the entire disclosure of which is incorporated herein by reference for all purposes.

### BACKGROUND

### 1. Technical Field

The present disclosure relates to an apparatus and method for supporting diagnosis of intraventricular hemorrhage and early death within a week in very low birth weight infants based on deep learning, which may more simply and rapidly diagnose the possibility of intraventricular hemorrhage and the possibility of early death within a week.

### 2. Background Art

According to the database registered in Korean very low birth weight infant network in 2019, 41.5% of very low birth weight infants (VLBWI) have experienced intraventricular hemorrhage (IVH), and 63.7% of them have showed severe IVH of grade 3 or higher.

IVH is associated with a poor prognosis and is a factor that increases the risk of death by up to three times. IVH in premature infants often occurs when several factors coincide or overlap, including changes in cerebral blood flow, dysregulation, and vulnerability of the periventricular germinal substrate. The condition is associated with prenatal and postnatal clinical scenarios.

In other words, IVH in very low birth weight infants occurs most frequently immediately after birth, and treatment options that may dramatically improve the prognosis of bleeding that has already occurred are still limited. Therefore, it is important to appropriately evaluate and manage the clinical status of infants before and after birth, and it is especially important to focus on IVH prevention.

Meanwhile, advances in big data analytics have led to the use of artificial intelligence (AI), such as machine learning or deep learning, in various fields.

AI has been used extensively in medicine to develop prediction models for IVH based on imaging techniques such as brain ultrasound or brain magnetic resonance imaging, but most of these studies have been conducted in adults.

For example, traditional statistical methods such as logistic regression have been used to identify risk factors in clinical studies of IVH based on initial data from the Korean Neonatal Network (KNN), a national cohort.

However, although KNN data has been used to analyze clinical factors in premature infants using Al technologies such as machine learning and deep learning, there is a limitation in that no study has been conducted to date that explicitly focuses on factors related to IVH.

### SUMMARY

The present disclosure is designed to solve the problems of the related art, and therefore the present disclosure is directed to providing an apparatus and method for supporting diagnosis of intraventricular hemorrhage and early death within a week in very low birth weight infants based on deep learning, which may diagnose the possibility of intraventricular hemorrhage and the possibility of early death within a week in advance using deep learning technology.

In addition, the present disclosure is directed to providing an apparatus and method for supporting diagnosis of intraventricular hemorrhage and early death within a week in very low birth weight infants based on deep learning, which may enable the diagnosis operation to be divisionally performed at three time points, namely before birth, immediately after birth, and one week after birth, in order to obtain and provide a treatment plan optimized for the diagnosis time.

The object of the present disclosure is not limited to the above, and other objects not mentioned herein will be clearly understood by those skilled in the art from the following disclosure.

In order to accomplish the above object, according to an embodiment of the present disclosure, there is provided a method for supporting diagnosis of intraventricular hemorrhage and early death within a week in very low birth weight infants based on deep learning by a device for prediction of intraventricular hemorrhage in very low birth weight infants, comprising: a data collection step of collecting and storing demographic information, maternal information, delivery information, neonatal information, disease information, vital signs at birth, vital signs for one week after birth, and intraventricular hemorrhage diagnosis result from a registered medical database; a data preprocessing step of generating a first feature value that includes demographic information and maternal information, a second feature value that includes delivery information, neonatal information, and vital signs at birth in addition to the first feature value, and a third feature value that includes vital signs for one week after birth and disease information in addition to the second feature value; a learning step of training a prenatal prediction model based on the first feature value and the intraventricular hemorrhage diagnosis result, training a birth prediction model based on the second feature value and the intraventricular hemorrhage diagnosis result, and training a postnatal prediction model based on the third feature value and the intraventricular hemorrhage diagnosis result; and a diagnosis step of, when a diagnosis target and diagnosis time are determined, selecting one prediction model based on the diagnosis time, analyzing medical information of the diagnosis target through the selected prediction model, and predicting and outputting the intraventricular hemorrhage diagnosis result.

The demographic information may include at least one of fetal sex and maternal age, the maternal information may include at least one of the number of pregnancies, in vitro fertilization, maternal diabetes, maternal hypertension, and clinical chorioamnionitis status, the delivery information may include at least one of a duration of premature rupture of membranes and mode of delivery, the neonatal information may include at least one of oxygen saturation, electrocardiogram, resuscitation status at delivery, gestational age, birth weight, 1-minute and 5-minute Apgar scores, pH, and base excess index, the disease information may include at least one of pulmonary hemorrhage, respiratory distress syndrome, and hypotension requiring drug treatment, and the vital signs may include at least one of oxygen saturation and electrocardiogram.

The medical information of the diagnosis target may include demographic information and maternal information when the diagnosis time is before birth, may include demographic information, maternal information, delivery information, neonatal information, and vital signs at birth when the diagnosis time is at birth, and may include demographic information, maternal information, delivery information, neonatal information, vital signs at birth, vital signs for one week after birth, and disease information when the diagnosis time is one week after birth.

Each of the first and postnatal prediction models may be implemented with one of LR (Logistic Regression with Ridge Regulation), RF (Random Forest), and XGB (extreme Gradient Boosting).

In order to accomplish the above object, according to another embodiment of the present disclosure, there is provided an apparatus for supporting diagnosis of intraventricular hemorrhage and early death within a week in very low birth weight infants based on deep learning, comprising: a data collection unit configured to collect and store demographic information, maternal information, delivery information, neonatal information, disease information, vital signs at birth, vital signs for one week after birth, and intraventricular hemorrhage diagnosis result from a registered medical database; a data preprocessing unit configured to generate a first feature value that includes demographic information and maternal information, a second feature value that includes delivery information, neonatal information, and vital signs at birth in addition to the first feature value, and a third feature value that includes vital signs for one week after birth and disease information in addition to the second feature value; a prediction model learning unit configured to train a prenatal prediction model based on the first feature value and the intraventricular hemorrhage diagnosis result, train a birth prediction model based on the second feature value and the intraventricular hemorrhage diagnosis result, and train a postnatal prediction model based on the third feature value and the intraventricular hemorrhage diagnosis result; and a diagnosis unit configured to, when a diagnosis target and diagnosis time are determined, select one prediction model based on the diagnosis time, analyze medical information of the diagnosis target through the selected prediction model, and predict and output the intraventricular hemorrhage diagnosis result.

The present disclosure may potentially reduce the incidence of permanent brain damage and premature death due to intraventricular hemorrhage in the early postnatal period by using deep learning techniques to diagnose intraventricular hemorrhage in very low birth weight infants.

In addition, by dividing the diagnosis into three time points—before birth, immediately after birth, and one week after birth—, it is possible to more easily and accurately establish and implement a treatment plan optimized for the diagnosis time.

### BRIEF DESCRIPTION OF THE DRAWINGS

FIG. 1 is a diagram for explaining a method for supporting diagnosis of intraventricular hemorrhage in very low birth weight infants based on deep learning according to an embodiment of the present disclosure.

FIG. 2 is a diagram for explaining an example of medical information used for learning and analyzing a prediction model according to an embodiment of the present disclosure.

FIG. 3 is a diagram for explaining an apparatus for supporting diagnosis of intraventricular hemorrhage and early death within a week in very low birth weight infants based on deep learning according to an embodiment of the present disclosure.

### DETAILED DESCRIPTION

Hereinafter, with reference to the attached drawings, preferred embodiments will be described in detail so that a person having ordinary skill in the art to which the present disclosure belongs can easily practice the present disclosure. However, when describing preferred embodiments of the present disclosure in detail, if a specific description of a related known function or configuration is judged to unnecessarily obscure the gist of the present disclosure, the detailed description will be omitted. In addition, the same reference numerals are used throughout the drawings for parts that have similar functions and actions.

In addition, throughout the specification, when a part is said to be ‘connected’ to another part, this includes not only the case where it is ‘directly connected’ but also the case where it is ‘indirectly connected’ with another element in between. Also, ‘including’ a certain component means that it can include other components rather than excluding other components unless specifically stated otherwise.

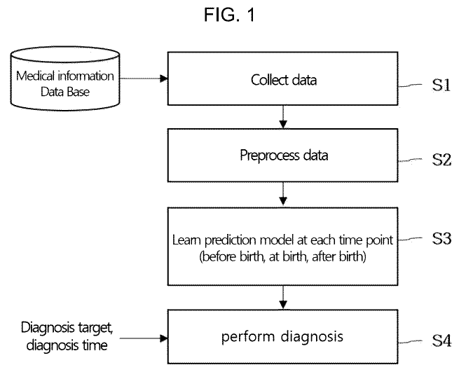

FIG. 1 is a diagram for explaining a method for supporting diagnosis of intraventricular hemorrhage in very low birth weight infants based on deep learning according to an embodiment of the present disclosure.

Referring to FIG. 1, the method of the present disclosure includes a data collection step (S1), a data preprocessing step (S2), a prediction model learning step (S3), and a diagnosis step (S4).

### Data Collection Step (S1)

Among very low birth weight infants with a gestational age of 23 weeks or more and a birth weight of 500 g or more but less than 1500 g, very low birth weight infants with mild intraventricular hemorrhage (IVH), very low birth weight infants with severe intraventricular hemorrhage, very low birth weight infants with early death within a week, and very low birth weight infants in normal condition are selected at certain proportions.

In addition, demographic information, maternal information, delivery information, neonatal information, disease information, vital signs at birth, vital signs for one week after birth, and intraventricular hemorrhage diagnosis results of the selected very low birth weight infants are collected from a pre-established medical information database (DB) and edited to generate a plurality of data sets, and store the plurality of data sets.

At this time, the demographic information may include at least one of fetal sex and maternal age, the maternal information may include at least one of the number of pregnancies, in vitro fertilization, maternal diabetes, maternal hypertension, and clinical chorioamnionitis status, the delivery information may include at least one of a duration of premature rupture of membranes and mode of delivery, the neonatal information may include at least one of oxygen saturation, electrocardiogram, resuscitation status at delivery, gestational age, birth weight, 1-minute and 5-minute Apgar scores, pH, and base excess index, the disease information may include at least one of pulmonary hemorrhage, respiratory distress syndrome, and hypotension requiring drug treatment, and the vital signs may include at least one of oxygen saturation and electrocardiogram, but need not be limited thereto.

In addition, the medical information database may be the Korea Neonatal Network (KNN) data set, which was established in May 2013 with the support of the Korean Society of Neonatology and the Korea Centers for Disease Control and Prevention, and in which about 70 out of about 100 neonatal intensive care units (NICUs) in Korea are participating. KNN prospectively registers about 2,000 to 2,400 very low birth weight infants (VLBWI) every year, and the data of registered VLBWI are utilized as national big data for various research purposes.

### Data Preprocessing Step (S2)

First, data preprocessing tasks such as missing value processing, outlier processing, duplicate data processing, and data format standardization are performed on the plurality of data sets.

After that, a first feature value that includes demographic information and maternal information is generated, a second feature value that further includes delivery information, neonatal information, and vital signs at birth in addition to the first feature value is generated, and a third feature value that further includes information one week after birth and disease information in addition to the second feature value is generated.

That is, in the present disclosure, all obtainable medical information is collected from the mother's period of preparation for birth to the first week postnatally, and then the collected data is classified into data at three time points, namely the situation immediately before birth, the situation within the first hour postnatally, and the first week postnatally, to enable learning and analyzing a prediction model.

### Prediction Model Learning Step (S3)

A plurality of first learning data that has the first feature value as an input condition and the intraventricular hemorrhage diagnosis result as an output condition is generated, a plurality of second learning data that has the second feature value as an input condition and the intraventricular hemorrhage diagnosis result as an output condition is generated, and a plurality of third learning data that has the third feature value as an input condition and the intraventricular hemorrhage diagnosis result as an output condition is generated.

In addition, a prenatal prediction model is machine-learned through a plurality of first learning data, a birth prediction model is machine-learned through a plurality of second learning data, and a postnatal prediction model is machine-learned through a plurality of third learning data.

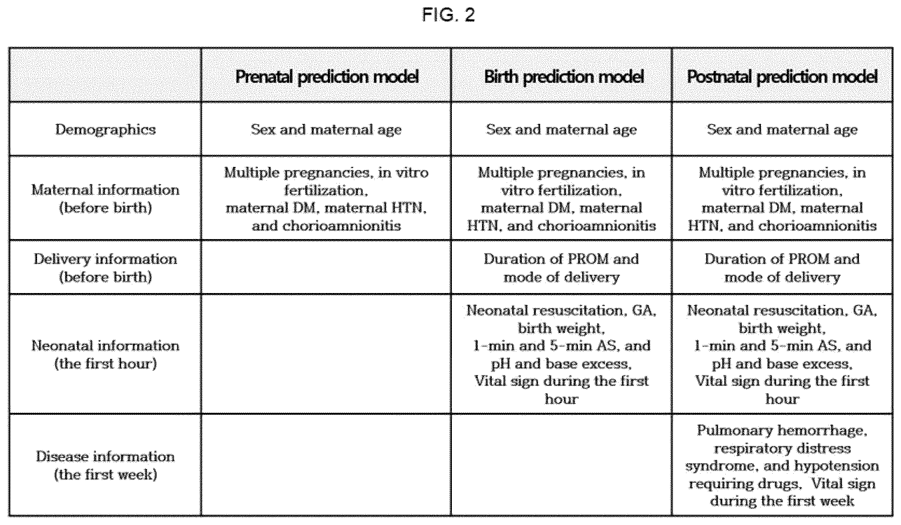

That is, three models, namely the prenatal prediction model, the birth prediction model, and the postnatal prediction model, which have different learning conditions as in FIG. 2, are generated, and prediction operations optimized for the current diagnosis time may be performed through these models.

More specifically, based on the plurality of data sets generated through the data collection step (S1), a plurality of first learning data having the medical information of very low birth weight infants before birth as the first input condition and the intraventricular hemorrhage diagnosis result as the output condition is generated, and then they are divided into training and validation sets in a ratio of 8:2 according to the numerical distribution.

The data sets are divided into training sets and validation sets while keeping the proportion of severe IVH/early death constant using hyperparameters within the split function.

The training set is used for model training and hyperparameter selection, and the validation set is used to evaluate the overall performance of the model.

To compare the baseline characteristics of very low birth weight infants in the training set and the validation set, bivariate t-tests are used for continuous variables and chi-square tests are used for categorical variables. The significance of group differences is evaluated using p-values and standardized mean differences.

Continuous predictor variables are normalized before being fed into the model using the min-max scaling method. This scales the range of numeric variable data points to be between 0 and 1 while maintaining their relative differences.

To address the imbalance of the target variable, the synthetic minority oversampling technique (SMOTE) is utilized, which promotes a balanced and efficient learning process by augmenting the data set by generating additional samples from the minority class in the derived data.

Training and tuning of the prediction model are performed using derived data via stratified k-fold cross-validation, and the cross-validation for the trained model is based on four iterations over the area under the receiver operating characteristic (AUROC).

In addition, the first or postnatal prediction model of the present disclosure may be built using classification machine learning algorithms such as, but not limited to, LR (Logistic Regression with Ridge Regulation), RF (Random Forest), and XGB (extreme Gradient Boosting).

### Diagnosis Step (S4)

Once the machine learning of the diagnostic model is complete, when the fetus or very low birth weight infants are selected as the diagnosis target and the diagnosis time is determined, one prediction model corresponding to the diagnosis time is selected among the pre-trained prediction models.

After acquiring medical information of the diagnosis target, the result of intraventricular hemorrhage diagnosis is predicted by analyzing the medical information through the selected prediction model, and the result is informed to the user audio-visually or provided to a preset external device.

At this time, the medical information of the diagnostician may also vary depending on the diagnosis time. If the diagnosis time is before birth, only demographic information and maternal information are included. If the diagnosis time is immediately after birth, delivery information, neonatal information, and vital signs at birth are further included in addition to demographic information and maternal information. If the diagnosis time is one week after birth, a third feature value that further includes demographic information, maternal information, delivery information, neonatal information, vital signs at birth, vital signs for one week after birth, and disease information is generated.

In this way, in the present disclosure, it is possible to monitor and track very low birth weight infants at three time points: before birth, immediately after birth, and one week after birth, and the results may be used to pre-train prediction models for each time point.

In addition, the prenatal prediction model may be used to predict premature birth or early death using prenatal data, which may help medical staff to prepare appropriate interventions in advance. The birth prediction model may be used to predict whether very low birth weight infants need intensive care using data up to 1 hour after delivery. Also, the postnatal prediction model may be used to prevent severe IVH or early death based on data up to 1 week postnatally.

That is, by accurately predicting the status of very low birth weight infants at the diagnosis time through a time-point prediction model and establishing an optimized treatment plan in advance based thereon, it is possible to prevent permanent brain damage or death of very low birth weight infants due to IVH. The treatment plan includes, for example, but not limited to, providing fluids and oxygen, a blood transfusion, a spinal tap, placing a tube or shunt in the brain to drain fluid, corticosteroids administered to a mother to lower the risk of IVH in her baby.

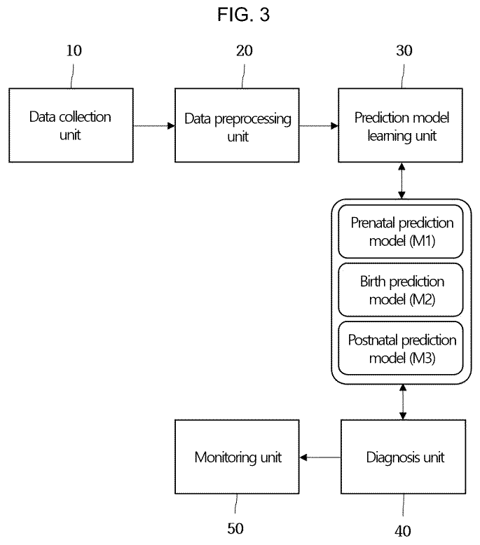

FIG. 3 is a diagram for explaining an apparatus for supporting diagnosis of intraventricular hemorrhage and early death within a week in very low birth weight infants based on deep learning according to an embodiment of the present disclosure.

Referring to FIG. 3, the apparatus of the present disclosure includes a data collection unit 10 configured to collect and store demographic information, maternal information, delivery information, neonatal information, disease information, vital signs at birth, vital signs for one week after birth, and intraventricular hemorrhage diagnosis result from a registered medical database, a data preprocessing unit 20 configured to generate a first feature value that includes demographic information and maternal information, a second feature value that includes delivery information, neonatal information, and vital signs at birth in addition to the first feature value, and a third feature value that includes vital signs for one week after birth and disease information in addition to the second feature value, a prediction model learning unit 30 configured to train a prenatal prediction model M1 based on the first feature value and the intraventricular hemorrhage diagnosis result, train a birth prediction model M2 based on the second feature value and the intraventricular hemorrhage diagnosis result, and train a postnatal prediction model M3 based on the third feature value and the intraventricular hemorrhage diagnosis result, a diagnosis unit 40 configured to, when a diagnosis target and diagnosis time are determined, select one prediction model based on the diagnosis time, analyze medical information of the diagnosis target through the selected prediction model, and predict and output the intraventricular hemorrhage diagnosis result, and a monitoring unit 50 configured to directly inform the intraventricular hemorrhage diagnosis result audio-visually or provide the same to a registered external device.

Although the present disclosure has been described with limited embodiments and drawings as above, the present disclosure is not limited to the embodiments described above, and those skilled in the art to which the present disclosure belongs can make various modifications and variations based on this description. Accordingly, the idea of the present disclosure should be understood only by the scope of the claims appended below, and all modifications equal or equivalent thereto are considered to fall within the scope of the idea of the present disclosure.

In addition, the apparatus and method according to the present disclosure can be implemented as a computer-readable code on a computer-readable recording medium. The computer-readable recording medium includes all types of recording devices that store data readable by a computer system. Examples of the recording medium include ROM, RAM, CD ROM, magnetic tape, floppy disk, optical data storage device, hard disk, flash drive, etc., and also include those implemented in the form of a carrier wave (e.g., transmission via the Internet). In addition, the computer-readable recording medium can be distributed over computer systems connected to a network, so that the computer-readable code can be stored and executed in a distributed manner.

What is claimed is:

1. A method for supporting diagnosis of intraventricular hemorrhage and early death within a week in a very low birth weight (VLBW) infant based on deep learning by a device for prediction of intraventricular hemorrhage in very low birth weight infants, the method comprising:
a data collection step of collecting and storing demographic information, maternal information, delivery information, neonatal information, disease information, vital signs at birth, vital signs for one week after birth, and intraventricular hemorrhage diagnosis result from a registered medical database;
a data preprocessing step of generating a first feature value that includes demographic information and maternal information, a second feature value that includes delivery information, neonatal information, and vital signs at birth in addition to the first feature value, and a third feature value that includes vital signs for one week after birth and disease information in addition to the second feature value;
a learning step of training a prenatal prediction model based on the first feature value and the intraventricular hemorrhage diagnosis result, training a birth prediction model based on the second feature value and the intraventricular hemorrhage diagnosis result, and training a postnatal prediction model based on the third feature value and the intraventricular hemorrhage diagnosis result; and
a diagnosis step of, when a diagnosis target and diagnosis time are determined, selecting one prediction model based on the diagnosis time, analyzing medical information of the diagnosis target through the selected prediction model, and predicting and outputting the intraventricular hemorrhage diagnosis result.

2. The method according to claim 1,
wherein the demographic information includes at least one of fetal sex and maternal age,
wherein the maternal information includes at least one of the number of pregnancies, in vitro fertilization, maternal diabetes, maternal hypertension, and clinical chorioamnionitis status,
wherein the delivery information includes at least one of a duration of premature rupture of membranes and mode of delivery,
wherein the neonatal information includes at least one of oxygen saturation, electrocardiogram, resuscitation status at delivery, gestational age, birth weight, 1-minute and 5-minute Apgar scores, pH, and base excess index,
wherein the disease information includes at least one of pulmonary hemorrhage, respiratory distress syndrome, and hypotension requiring drug treatment, and
wherein the vital sign includes at least one of oxygen saturation and electrocardiogram.

3. The method according to claim 1, wherein the medical information of the diagnosis target includes demographic information and maternal information when the diagnosis time is before birth, includes demographic information, maternal information, delivery information, neonatal information, and vital signs at birth when the diagnosis time is at birth, and includes demographic information, maternal information, delivery information, neonatal information, vital signs at birth, vital signs for one week after birth, and disease information when the diagnosis time is one week after birth.

4. The method according to claim 1, wherein each of the first and postnatal prediction models is implemented with one of LR (Logistic Regression with Ridge Regulation), RF (Random Forest), and XGB (extreme Gradient Boosting).

5. An apparatus for supporting diagnosis of intraventricular hemorrhage and early death within a week in a very low birth weight (VLBW) infant based on deep learning, the apparatus comprising:
a data collection unit configured to collect and store demographic information, maternal information, delivery information, neonatal information, disease information, vital signs at birth, vital signs for one week after birth, and intraventricular hemorrhage diagnosis result from a registered medical database;
a data preprocessing unit configured to generate a first feature value that includes demographic information and maternal information, a second feature value that includes delivery information, neonatal information, and vital signs at birth in addition to the first feature value, and a third feature value that includes vital signs for one week after birth and disease information in addition to the second feature value;
a prediction model learning unit configured to train a prenatal prediction model based on the first feature value and the intraventricular hemorrhage diagnosis result, train a birth prediction model based on the second feature value and the intraventricular hemorrhage diagnosis result, and train a postnatal prediction model based on the third feature value and the intraventricular hemorrhage diagnosis result; and
a diagnosis unit configured to, when a diagnosis target and diagnosis time are determined, select one prediction model based on the diagnosis time, analyze medical information of the diagnosis target through the selected prediction model, and predict and output the intraventricular hemorrhage diagnosis result.
